(19) United States
(12) Patent Application Publication
(10) Pub. No.: US 2024/0237985 A1
(43) Pub. Date: Jul. 18, 2024

(54) ANVIL AND DELIVERY SYSTEM ASSEMBLY WITH AUTOMATIC RETAINING SUTURE RELEASE

(71) Applicant: Covidien LP, Mansfield, MA (US)

(72) Inventors: Yanli Wan; Yishuai Xu, Shanghai (CN); Jinhang Liu, Shanghai (CN); Yanjun Jiao, Shanghai (CN); Xiliang Zhang, Shanghai (CN)

(21) Appl. No.: 18/563,122

(22) PCT Filed: May 24, 2021
(86) PCT No.: PCT/CN2021/095510

§ 371 Date: Nov. 21, 2023

(51) Int. Cl. A61B 17/115; A61B 17/00

(52) U.S. Cl. CPC A61B 17/1155; A61B 2017/00477

(57) ABSTRACT

An anvil and delivery system assembly includes an anvil and a delivery system assembly. The delivery system includes a flexible guide member, an adapter, a tie down suture, and a retaining suture. The guide member is coupled to the center rod with the adapter. The tie down suture is secured to the adapter and to the anvil head to retain the anvil head in the tilted position during anvil delivery. The retaining suture is coupled to the anvil head via the tie down suture such that release of the tie down suture automatically releases the retaining suture from the anvil head.

### FIELD

The technology is generally related to anvil assemblies for surgical stapling devices and, more particularly, to an anvil and delivery system assembly for delivering an anvil to an anastomotic site prior to a surgical anastomosis procedure.

### BACKGROUND

Trans-oral delivery systems for delivering an anvil to a surgical site, e.g., the stomach, prior to performance of a surgical anastomosis procedure are known. Typically, the delivery system includes flexible guide member that is releasably coupled to a center rod of the anvil, a retaining suture that is coupled to an anvil head of the anvil, and a tie down suture that is secured to the anvil head and to an adapter that is coupled to the flexible guide member to secure the anvil head in a tilted position.

When the anvil is delivered trans-orally to an anastomotic site, e.g., the stomach, the guide member is fed through the esophagus into the stomach and through openings in the stomach and the abdominal wall. When the guide member exits through the opening in the abdominal wall, the guide member is grasped by a clinician and pulled through the abdominal wall to pull the anvil through the esophagus and into the stomach. As the anvil is being pulled into the stomach, the clinician grasps the retaining suture which extends from the mouth of the patient and may be used by a clinician to dislodge the anvil if the anvil becomes stuck during delivery of the anvil and/or to retrieve the anvil in the event of a patient emergency, e.g., cardiac arrest.

After the anvil is delivered to the anastomotic site, the flexible guide member is disconnected from the center rod of the anvil by cutting the tie down suture, and the anvil is coupled to a circular stapling device to perform the anastomosis procedure. In certain situations, the retaining suture may become trapped between the anvil and the circular stapling device and subsequently stapled to the anastomotic site.

A continuing need exists in the art for an anvil and delivery system assembly that ensures that the retrieval suture does not interfere with formation of the anastomosis.

### SUMMARY

Aspects of this disclosure are directed to an anvil and delivery system assembly that can be used to delivery an anvil to a surgical site during a surgical anastomosis procedure. The delivery system includes a flexible guide member, an adapter, a tie down suture, and a retaining suture. The tie down suture is secured to an anvil head of the anvil to retain the anvil head in a tilted position during anvil delivery. The retaining suture is coupled to the anvil head via the tie down suture such that release of the tie down suture automatically releases the retaining suture from the anvil head.

Aspects of this disclosure are directed to an anvil and delivery system assembly including an anvil and a delivery system. The anvil includes an anvil head and a center rod. The anvil head is secured to the center rod by a pivot member and is movable between a first tilted position, an operative position, and a second tilted position. The delivery system includes a flexible guide member, an adapter, a tie down suture, and a retaining suture. The flexible guide member is coupled to the center rod of the anvil with the adapter. The tie down suture is secured to the anvil head and to the adapter to retain the anvil head in the first tilted position. The retaining suture is secured to the anvil head via the tie down suture such that release of the tie down suture from the anvil head automatically releases the retaining suture from the anvil head.

In aspects of this disclosure, the anvil head includes a body defining first and second openings, and the tie down suture extends through the first opening in the anvil head and from the second opening of the anvil head to secure the tie down suture to the anvil head.

In some aspects of this disclosure, the body of the anvil head defines a third opening, and the retaining suture extends into the third opening, is wound about the tie down suture, and extends back through the third opening to secure the retaining suture to the anvil head.

In certain aspects of this disclosure, the adapter includes a distal connector that is received within the flexible guide member to frictionally secure the adapter to the flexible guide member.

In aspects of this disclosure, the tie down suture includes first and second ends that are compressed between the distal connector of the adapter and the flexible guide member.

In some aspects of this disclosure, the delivery system includes a reel, and a portion of the retaining suture is supported within the reel and can be selectively fed from the reel.

In certain aspects of this disclosure, the distal connector includes ribs that engage the flexible guide member to secure the adapter within the flexible guide member.

In aspects of this disclosure, the adapter includes a proximal connector that is received within the center rod of the anvil.

In some aspects of this disclosure, the center rod includes a distal portion that is coupled to the anvil head by the pivot member and a proximal portion that includes flexible legs that define a longitudinal bore.

In certain aspects of this disclosure, the longitudinal bore is dimensioned to releasably receive a trocar of a stapling device to releasably secure the anvil to the stapling device.

Other aspects of this disclosure are directed to an anvil and delivery system assembly that includes an anvil and a delivery system. The anvil includes an anvil head and a center rod. The anvil head is secured to the center rod by a pivot member and is movable between a tilted position and an operative position. The delivery system includes a flexible guide member, a tie down suture, and a retaining suture. The flexible guide member is coupled to the center rod of the anvil. The tie down suture is secured to the anvil head and to the flexible tube to retain the anvil head in the tilted position. The retaining suture is secured to the anvil head via the tie down suture such that release of the tie down suture from the anvil head automatically releases the retaining suture from the anvil head.

In aspects of this disclosure, the proximal connector of the adapter includes a rib that is received between the flexible legs of the center rod to prevent rotation of the center rod in relation to the adapter.

Other features of the disclosure will be appreciated from the following description.

### BRIEF DESCRIPTION OF DRAWINGS

Various aspects of the disclosure are described herein below with reference to the drawings, wherein:

FIG. 1 is a side perspective view of a Prior Art circular stapling device;

FIG. 1A is a side perspective view of an anvil and delivery system assembly including anvil for use with the stapling device of FIG. 1 with an anvil head of the anvil in a tilted position;

FIG. 2 is an enlarged view of the indicated area of detail shown in FIG. 1;

FIG. 3A is a side perspective view of the anvil and anvil delivery system shown in FIG. 1 with the anvil delivery system coupled to the anvil and the anvil head assembly of the anvil in the tilted position;

FIG. 3B is aside perspective view of an adapter and flexible guide member of the anvil delivery system shown in FIG. 1;

FIG. 4 is an enlarged view of the indicated area of detail shown in FIG. 3A;

FIG. 5 is a side perspective view of the anvil shown in FIG. 4 showing the interconnection between the tie down suture and the guide suture;

FIG. 6 is a view of the anvil and the anvil delivery system as the anvil is delivered to an anastomosis site within the body of a patient;

FIG. 7 is a side perspective view of the anvil and anvil delivery system as the anvil delivery system is released from the anvil;

FIG. 8 is a side perspective view of the anvil and delivery system assembly with the delivery system released from the anvil; and

FIG. 9 is a side perspective view of the distal portion of the circular stapling device with the anvil attached to the anvil retainer of the circular stapling device.

### DETAILED DESCRIPTION

The disclosed anvil and delivery system assembly will now be described in detail with reference to the drawings in which like reference numerals designate identical or corresponding elements in each of the several views. However, it is to be understood that the aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device in its customary fashion, while the term “distal” is used generally to refer to that portion of the device that is farther from the clinician during use of the device in its customary fashion. In addition, the term “clinician” is used generally to refer to medical personnel including doctors, nurses, and support personnel.

This disclosure is directed to an anvil and delivery system assembly that can be used to delivery an anvil to a surgical site during a surgical anastomosis procedure. The anvil and delivery system assembly includes an anvil and a delivery system. The anvil an anvil head that is coupled to a center rod by a pivot member that facilitates pivotal movement of the anvil head in relation to the center rod between tilted and operative positions. The delivery system includes a flexible guide member, an adapter, a tie down suture, and a retaining suture. The guide member is coupled to the center rod with the adapter. The tie down suture is secured to the adapter and to the anvil head to retain the anvil head in the tilted position during anvil delivery. The retaining suture is coupled to the anvil head via the tie down suture such that release of the tie down suture automatically releases the retaining suture from the anvil head.

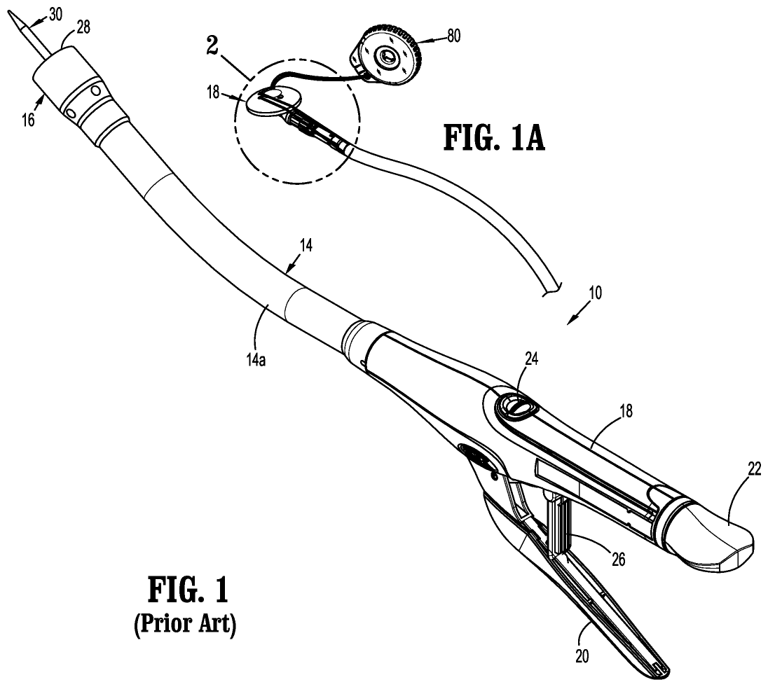

FIG. 1 illustrates a surgical stapling device that is configured for use with an anvil having a tiltable anvil head. Although the anvil and delivery system assembly of the disclosure will be described with reference to a surgical stapling device 10, it is envisioned that the anvil assemblies and delivery systems may be modified for use with alternative surgical stapling devices.

The surgical stapling device 10 includes a handle assembly 12, an elongated body 14 having a curved elongate outer tube 14a, a shell assembly 16, and an anvil 18. The length, shape and/or the diameter of the body portion 14 and the shell assembly 16 may be modified to suit a particular surgical procedure.

The handle assembly 12 includes a stationary handle 18, a firing trigger 20, a rotatable approximation knob 22, and an indicator 24. A trigger lock 26 is coupled to the handle assembly 12 by a pivot member (not shown) and is manually movable from an unlocked position to a locked position to prevent inadvertent firing of the stapling device 10. The indicator 24 is positioned on the stationary handle 18 and includes indicia, e.g., color coding, alpha-numeric labeling, etc., to identify to a clinician when the stapling device 10 is approximated and is in a fire-ready position.

The shell assembly 16 of the tool assembly includes a staple cartridge 28 that supports staples (not shown). The stapling device 10 includes a trocar assembly 30 that extends from the elongate body 14 and through the shell assembly 16. The trocar assembly 30 is adapted to be releasably coupled to the anvil 18. For a more detailed discussion of an exemplary surgical stapling device, please refer to U.S. Pat. No. 7,364,060.

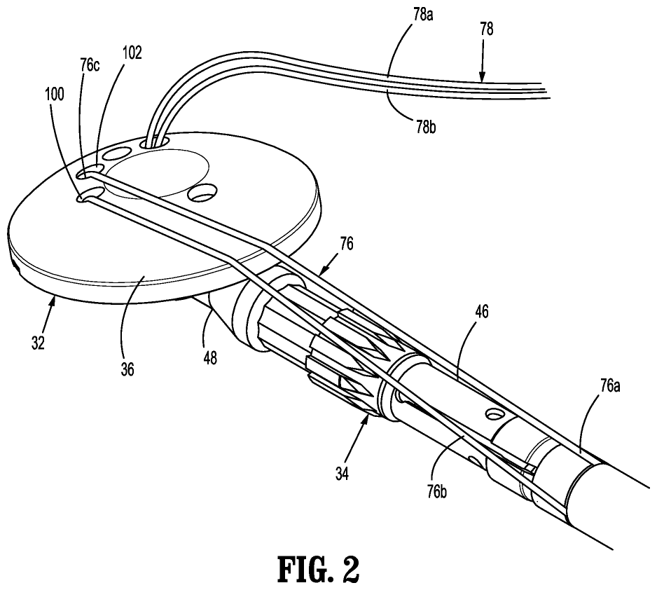

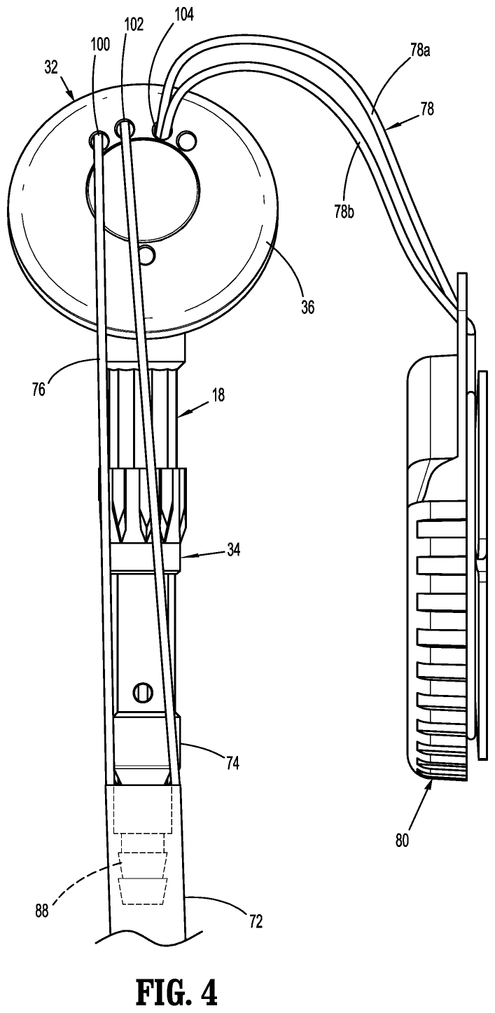

FIG. 2 illustrates the anvil 18 which includes an anvil head 32 and a center rod 34. The anvil head 32 includes a body 36, an anvil plate 38 (FIG. 5), and a central post 40 (FIG. 5). The body 36 of the anvil head 32 defines a circular cavity 42 (FIG. 5) that receives the anvil plate 38. The anvil plate 38 has a circular configuration and defines a plurality of staple forming pockets 44 that are positioned about the circular cavity 42. The post 40 is secured to, or integrally formed with, the body 36 and is centrally positioned within the circular cavity 42.

The center rod 34 includes a proximal portion 46 and a distal portion 48. The proximal portion of the center rod 34 defines a clevis 50 (FIG. 5) that is secured to the post 40 of the anvil head 32 by a pivot member 52 (FIG. 5) to facilitate pivotable movement of the anvil head 32 in relation to the center rod 34 between tilted and operative positions. The proximal portion 46 of the center rod 34 includes flexible legs 56 that define a longitudinal bore 58 (FIG. 5) that receives a distal portion of the trocar assembly 30 (FIG. 1) to releasably secure the anvil 18 to the trocar assembly 30. The center rod 34 also includes a biasing mechanism that is positioned to urge the anvil head 32 initially the operative position and subsequently towards a second tilted position. U.S. Pat. Nos. 10,881,410, 10,779,833, and 9,861.367 disclose anvil assemblies suitable for use with the anvil delivery system to be described herein.

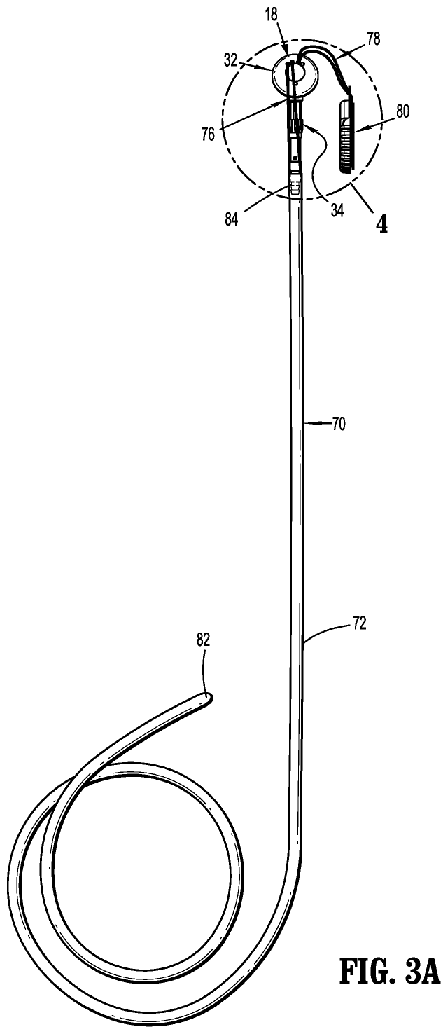

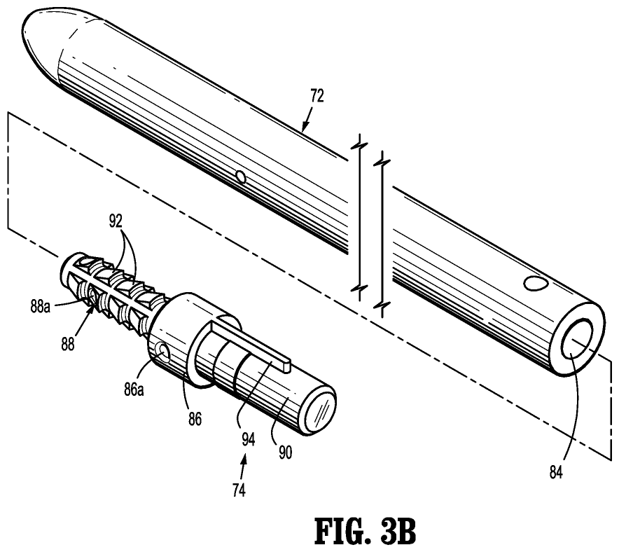

FIGS. 3A-5 illustrate the anvil delivery system coupled to the anvil 18. The anvil delivery system is shown generally as delivery system 70 and includes a flexible guide member 72, an adapter 74, a tie down suture 76, a retaining suture 78, and a reel 80. The flexible guide member 72 includes a blunt distal portion 82 and an open proximal portion 84. The adapter 74 (FIG. 3B) includes a central hub 86 that defines a through bore 86a, a distal connector 88, and a proximal connector 90. The distal connector 88 is received in the open proximal portion 84 of the flexible guide member 72 and includes ribs or teeth 92 that engage an inner wall of the flexible guide member 72 to frictionally secure the adapter 74 to the proximal portion 84 of the flexible guide member 72. The distal connector 88 also defines a through bore 88a. The proximal connector 90 of the adapter 74 is received within the proximal portion 46 of the center rod 34 of the anvil 18. In aspects of the disclosure, the proximal connector 90 of the adapter 74 includes a longitudinal rib 94 that is received between the flexible legs 56 of the center rod 34 to prevent rotation of the center rod 34 in relation to the adapter 74.

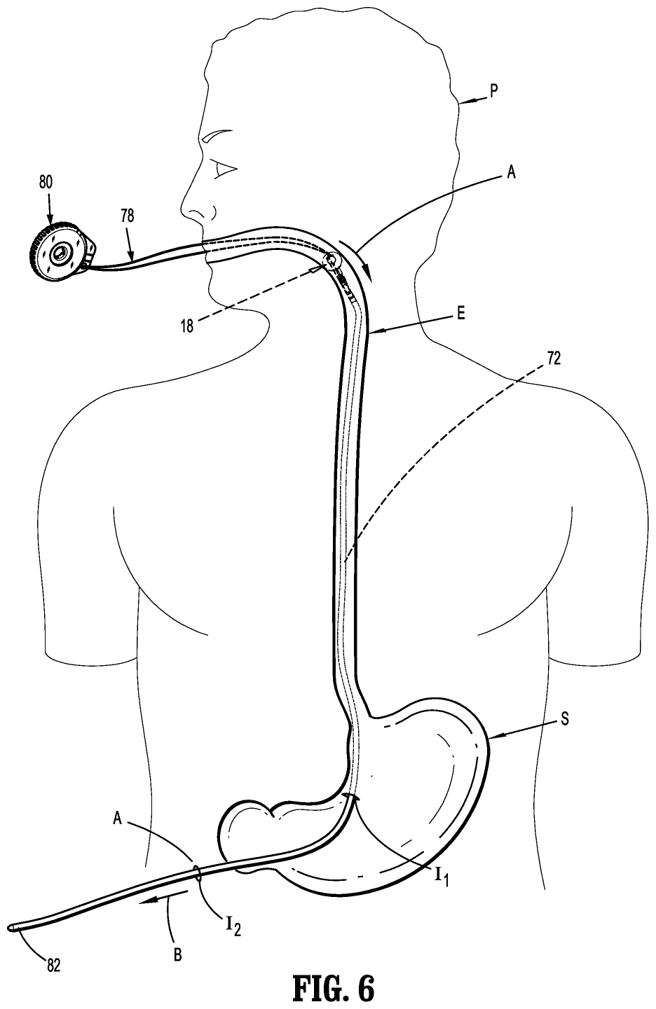

The tie down suture 76 includes first and second ends 76a and 76b and a central portion 76c. The tie down suture 76 is inserted through a first opening 100 in the body 36 of the anvil head 32 and exits a second opening 102 in the body 36 of the anvil head 32. The first and second ends 76a and 76b of the tie down suture 76 are compressed between the distal portion 88 of the adapter 74 and an inner surface of the flexible guide member 72 (FIG. 7) within the open proximal end 84 of the flexible guide member 72. The tie down suture 76 is maintained in tension to hold the anvil head 32 in the tilted position and to retain the proximal connector 90 of the adapter 74 within the proximal portion 46 of the center rod 34. In an alternate aspect of the disclosure, the tie down suture 76 can be inserted through the transverse bore 86a in the adapter 74 to secure the anvil head 32 of the anvil 18 to the adapter 74 and to secure the center rod 34 to the adapter 74.

The retaining suture 78 is inserted through a third opening 104 in the body 36 of the anvil head 32 and includes a central portion 78c that is wound about the central portion 76c of the tic down suture 76 (FIG. 5). The retaining suture 78 is inserted back through the third opening 104 such that the central portion 76c of the tie down suture 76 couples the retaining suture 78 to the anvil head 32 of the anvil 18. The retaining suture 78 includes first and second ends 78a and 78b that extend from the central portion 78c of the retaining suture 78 and are received within the reel 80. The reel 80 includes a length of retaining suture 78 and allows the retaining suture 78 to be selectively fed from the reel 80 as the anvil 18 is delivered to an anastomotic site within a body cavity, e.g., the stomach.

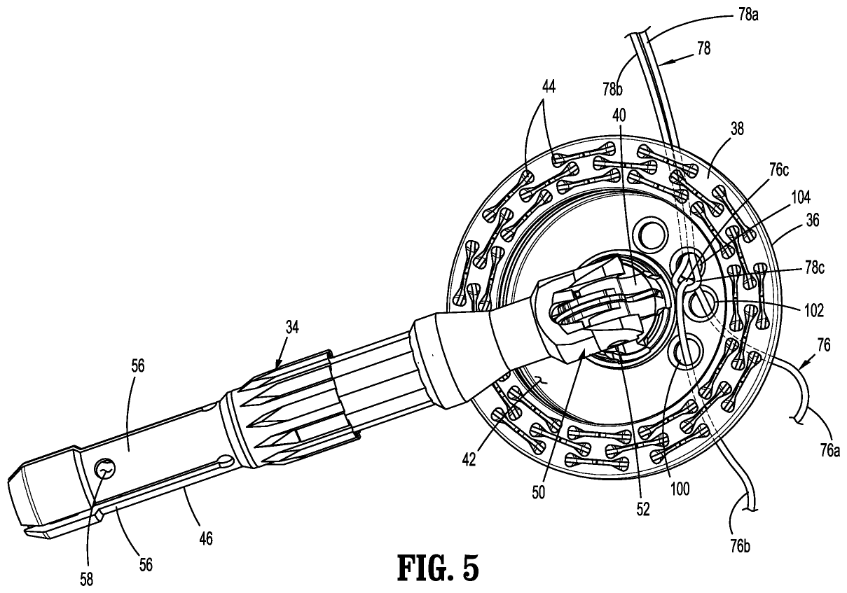

FIG. 6 illustrates delivery of the anvil to an anastomotic site, e.g., the stomach “S”, of a patient “P” during an anastomosis procedure. During an anastomosis procedure, the flexible guide member 72 of the delivery system 70 is inserted through the patient's mouth “M” and fed through the esophagus “E” of the patient “P” in the direction of arrow “A” into the stomach “S”. After the distal portion 82 of the flexible guide member 72 is received within the stomach “S”, the distal portion 82 of the flexible guide member 72 is inserted through an incision “I₁” in the stomach “S” and through an incision “I₂” in the abdominal wall “A” of the patient “P” in the direction of arrow “B”. When the distal portion 82 of the flexible guide member 72 is positioned externally of the patient “P”, the distal portion 82 of the flexible guide member 72 is grasped by the clinician and pulled to move the anvil 18 through the esophagus “E” and into the stomach “S”. It is noted that as the anvil 18 is fed into the patient “P”, the retaining suture 78 is fed from the reel 80 into the esophagus “E” and stomach “S”.

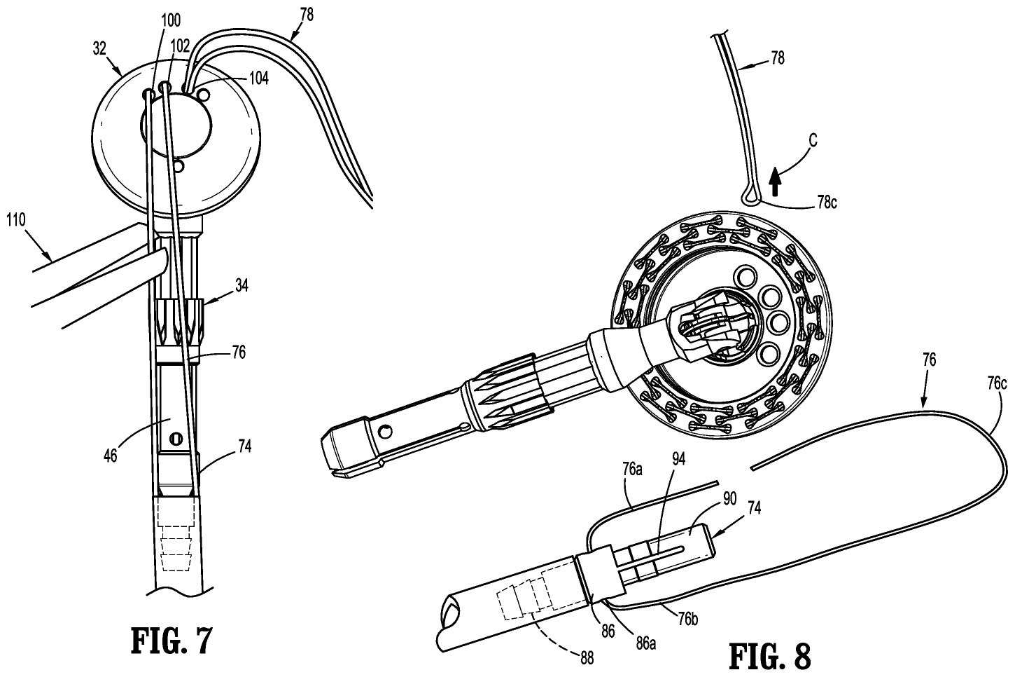

FIGS. 7 and 8 illustrate the anvil 18 as it would be in the stomach “S” of the patient “P”. Once the anvil 18 is delivered to the stomach “S”, the anvil 18 can be released from the adapter 74 of the delivery system 70 by cutting the tie down suture 76 with a scissor 110 and withdrawing the flexible guide member 72 from the patient “P” through the incisions “I₁” and “I₂” in the patient “P”. As described above, the adapter 74 is fixedly coupled to the flexible guide member 72 of the delivery system 70. When the flexible guide member 72 is withdrawn from the patient “P”, the adapter 74 and the tie down suture 76 which is clamped between the adapter 74 and the flexible guide member 72, are withdrawn from the patient “P”. When the tie down suture 76 is withdrawn from the openings 100 and 102 of the body 36 of the anvil head 32 of the anvil 18, the tie down suture 76 is uncoupled from the retaining suture 78 to release the retaining suture 78 from the anvil head 32 (FIG. 8). At this point, the retaining suture 78 can be removed from the patient “P” in the direction of arrow “C” in FIG. 8 by pulling on the reel 80 to withdraw the retaining suture 78 through the esophagus “E”.

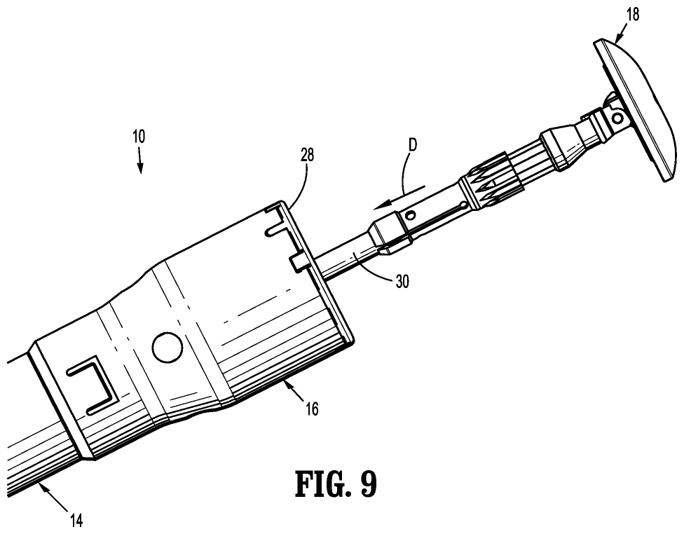

After the delivery system 70 is removed from the patient “P”, the anvil 18 can be coupled to the trocar assembly 30 of the stapling device 10 by inserting the trocar assembly 30 into the proximal portion 46 of the center rod 34 in the direction of arrow “D” in FIG. 9. At this point, the stapling device 10 can be operated in its customary manner to complete the anastomosis procedure without any concern of the retaining suture 78 becoming staples to the anastomotic site.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. An anvil and delivery system assembly comprising:
an anvil including an anvil head and a center rod, the anvil head secured to the center rod by a pivot member and movable between a first tilted position, an operative position, and a second tilted position; and
a delivery system including a flexible guide member, an adapter, a tie down suture, and a retaining suture, the flexible guide member coupled to the center rod of the anvil with the adapter, the tie down suture secured to the anvil head and to the adapter to retain the anvil head in the first tilted position, and the retaining suture secured to the anvil head via the tie down suture such that release of the tie down suture from the anvil head automatically releases the retaining suture from the anvil head.

2. The anvil and delivery system assembly of claim 1, wherein the anvil head includes a body defining first and second openings, the tie down suture extending through the first opening in the anvil head and from the second opening of the anvil head to secure the tie down suture to the anvil head.

3. The anvil and delivery system assembly of claim 2, wherein the body of the anvil head defines a third opening, and the retaining suture extends into the third opening, is wound about the tie down suture, and extends back through the third opening to secure the retaining suture to the anvil head.

4. The anvil and delivery system assembly of claim 3, wherein the adapter includes a distal connector that is received within the flexible guide member to frictionally secure the adapter to the flexible guide member.

5. The anvil and delivery system assembly of claim 4, wherein the tie down suture includes first and second ends, the first and second ends compressed between the distal connector of the adapter and the flexible guide member.

6. The anvil and delivery system assembly of claim 5, wherein the delivery system includes a reel, and a portion of the retaining suture is supported within the reel and can be selectively fed from the reel.

7. The anvil and delivery system assembly of claim 4, wherein the distal connector includes ribs that engage the flexible guide member to secure the adapter within the flexible guide member.

8. The anvil and delivery system assembly of claim 4, wherein the adapter includes a proximal connector that is received within the center rod of the anvil.

9. The anvil and delivery system assembly of claim 1, wherein the center rod includes a distal portion that is coupled to the anvil head by the pivot member and a proximal portion that includes flexible legs that define a longitudinal bore, the longitudinal bore dimensioned to releasably receive a trocar of a stapling device to releasably secure the anvil to the stapling device.

10. An anvil and delivery system assembly comprising:
an anvil including an anvil head and a center rod, the anvil head secured to the center rod by a pivot member and movable between a tilted position and an operative position; and
a delivery system including a flexible guide member, a tie down suture, and a retaining suture, the flexible guide member coupled to the center rod of the anvil, the tie down suture secured to the anvil head and to the flexible tube to retain the anvil head in the tilted position, and the retaining suture secured to the anvil head via the tie down suture such that release of the tie down suture from the anvil head automatically releases the retaining suture from the anvil head.

11. The anvil and delivery system assembly of claim 10, wherein the anvil head includes a body defining first and second openings, the tie down suture extending through the first opening in the anvil head and from the second opening of the anvil head to secure the tie down suture to the anvil head.

12. The anvil and delivery system assembly of claim 11, wherein the body of the anvil head defines a third opening, and the retaining suture extends into the third opening, is wound about the tie down suture, and extends back through the third opening to secure the retaining suture to the anvil head.

13. The anvil and delivery system assembly of claim 13, further including an adapter coupling the flexible guide member to the center rod of the anvil.

14. The anvil and delivery system assembly of claim 13, wherein the adapter includes a distal connector that is received within the flexible guide member to frictionally secure the adapter to the flexible guide member.

15. The anvil and delivery system assembly of claim 14, wherein the tie down suture includes first and second ends, the first and second ends compressed between the distal connector of the adapter and the flexible guide member.

16. The anvil and delivery system assembly of claim 15, wherein the delivery system includes a reel, and a portion of the retaining suture is supported within the reel and can be selectively fed from the reel.

17. The anvil and delivery system assembly of claim 14, wherein the distal connector includes ribs that engage the flexible guide member to secure the adapter within the flexible guide member.

18. The anvil and delivery system assembly of claim 17, wherein the adapter includes a proximal connector that is received within the center rod of the anvil.

19. The anvil and delivery system assembly of claim 12, wherein the center rod includes a distal portion that is coupled to the anvil head by the pivot member and a proximal portion that includes flexible legs that define a longitudinal bore, the longitudinal bore dimensioned to releasably receive a trocar of a stapling device to releasably secure the anvil to the stapling device.

20. The anvil and delivery system assembly of claim 19, wherein the proximal connector of the adapter includes a rib that is received between the flexible legs of the center rod to prevent rotation of the center rod in relation to the adapter.
